(19) United States
(12) Patent Application Publication
(10) Pub. No.: US 2013/0237579 A1
(43) Pub. Date: Sep. 12, 2013

(54) STABLE PHARMACEUTICAL COMPOSITIONS COMPRISING ONE OR MORE HMG-COA REDUCTAS INHIBITIORS

(71) Applicants: Timothy Stanley, Hertfordshire (GB); Isabel Delgado, Barcelona (ES); Estela Llorens, Barcelona (ES); Eva Munoz, Barcelona (ES)

(72) Inventors: Timothy Stanley, Hertfordshire (GB); Isabel Delgado, Barcelona (ES); Estela Llorens, Barcelona (ES); Eva Munoz, Barcelona (ES)

(21) Appl. No.: 12/668,544

(22) PCT Filed: Jul. 11, 2008
(86) PCT No.: PCT/GB08/50559

§ 371 Date: Mar. 14, 2013

(30) Foreign Application Priority Data
Jul. 13, 2007 (GB) 0713707.8

(51) Int. Cl. A61K 31/405

(52) U.S. Cl. CPC A61K 31/405
USPC 514/419

(57) ABSTRACT

The present invention relates to stable pharmaceutical compositions comprising one or more HMG-CoA reductase inhibitors, processes for preparing the stable compositions and uses for the compositions. The stable pharmaceutical compositions of the invention may be used, in particular, for the treatment of hyperlipoproteinemia and atherosclerosis.

### CROSS-REFERENCE TO RELATED APPLICATION(S)

This application is a Section 371 National Stage Application of International No. PCT/GB2008/050559, filed 11 Jul. 2008 and published as WO 2009/010787 A2 on 22 Jan. 2009, which claims priority from the Great Britain Application 0713707.8, filed 13 Jul. 2009, the contents of which are incorporated herein in their entirety for all purposes.

### FIELD OF THE INVENTION

The present invention relates to stable pharmaceutical compositions comprising one or more HMG-CoA reductase inhibitors, processes for preparing the stable compositions and uses for the compositions. The stable pharmaceutical compositions of the invention may be used, in particular, for the treatment of hyperlipoproteinemia and atherosclerosis.

### BACKGROUND OF THE INVENTION

HMG-CoA reductase inhibitors such as fluvastatin, pravastatin, lovastatin, simvastatin, atorvastatin, cerivastatin and rosuvastatin are used commercially as antihypercholesterolemic agents for the treatment of hyperlipoproteinemia and atherosclerosis. However, HMG-CoA reductase inhibitors and structurally related drugs (this class of compounds is commonly referred to as ‘statins’) contain a dihydroxyheptenoic acid moiety and it has been found that the statins are very unstable and are prone to degradation when formulated into pharmaceutical compositions.

Consequently, as a stable pharmaceutical composition is essential to obtain regulatory approval to market a drug, there have been many published attempts in the art to manufacture pharmaceutical compositions containing one or more statins wherein the pharmaceutical composition has acceptable stability.

The method which has typically been used to stabilise the pharmaceutical compositions comprising the statin is the use of an alkaline agent in the composition such that the pH of the composition when dispersed in water would be approximately pH 8 or higher. The pH of the composition is kept high to protect the statin against pH related degradation as it has been theorised that the instability of the statin compounds is due to the extreme lability of the dihydroxyheptenoic acid moiety at neutral or acidic pH.

For example, pharmaceutical compositions comprising HMG-CoA reductase inhibitors wherein the pharmaceutical compositions have enhanced stability due to the presence of alkaline agents and/or buffering agents have been disclosed in patent applications EP 0547000, EP 0336298, EP 1292293, WO 94/16693, WO 01/76566, WO 06/006021 and WO 00/35425.

Typical alkaline agents or mediums disclosed in these prior art documents are inorganic alkaline agents such as sodium carbonate; sodium bicarbonate; potassium carbonate; potassium bicarbonate; calcium carbonate; calcium bicarbonate; magnesium carbonate; magnesium bicarbonate; sodium hydroxide; potassium hydroxide; calcium hydroxide; lithium hydroxide; ammonium hydroxide; aluminium hydroxide; magnesium oxide; magnesium hydroxide; magnesium aluminium hydroxide; magnesium aluminium silicate; phosphate salts (e.g. sodium, potassium or calcium dibasic phosphate, tribasic calcium phosphate or trisodium phosphate); and mixtures thereof. Polymeric amides, such as polyvinylpyrrolidine, and organic amines, such as 1-adamantyl amine, tris(hydroxymethyl)ethylenediamine, triethanolamine, meglumine or L-arginine, have also been disclosed as stabilising alkaline agents.

Of the above mentioned alkaline agents, the most preferred agents used in the prior art to stabilise pharmaceutical compositions comprising HMG-CoA reductase inhibitors are the inorganic carbonate and bicarbonate salts. However, the use of alkaline agents in these formulations can cause problems for patients taking the pharmaceutical composition, particularly for patients with a damaged gastric mucous membrane.

### SUMMARY AND DETAILED DESCRIPTION OF THE INVENTION

The present invention provides a stable pharmaceutical composition comprising one or more HMG-CoA reductase inhibitors, wherein the pharmaceutical composition has enhanced stability. The pharmaceutical compositions of the current invention have enhanced stability over extended periods of time, e.g. whereby at least 95% of the initial amount of the active drug is still active after 2 years at ambient conditions.

One embodiment of the first aspect of the present invention is a stable pharmaceutical composition comprising one or more HMG-CoA reductase inhibitors, wherein the pharmaceutical composition does not include an alkaline agent. Surprisingly, a stable pharmaceutical composition has been prepared comprising one or more HMG-CoA reductase inhibitors, wherein the pharmaceutical composition does not contain an alkaline agent.

The term ‘HMG-CoA reductase inhibitor’ includes lactone derivatives of or ring-open forms of 7-substituted-3,5-dihydroxyheptanoic acids or 7-substituted-3,5-dihydroxy-heptenoic acids or their pharmaceutically acceptable salts.

The term ‘alkaline agent’ includes any agent which causes the pH of the composition when dispersed in water to be approximately pH 8 or higher. Typical alkaline agents are inorganic alkaline agents such as sodium carbonate; sodium bicarbonate; potassium carbonate; potassium bicarbonate; calcium carbonate; calcium bicarbonate; magnesium carbonate;

magnesium bicarbonate; sodium hydroxide; potassium hydroxide; calcium hydroxide; lithium hydroxide; ammonium hydroxide; aluminium hydroxide; magnesium oxide; magnesium hydroxide; magnesium aluminium hydroxide; magnesium aluminium silicate; and phosphate salts (e.g. sodium, potassium or calcium dibasic phosphate, tribasic calcium phosphate or trisodium phosphate). Typical organic alkaline agents are polymeric amides, such as polyvinylpyrrolidine; and amines, such as 1-adamantyl amine, tris(hydroxymethyl)ethylenediamine, triethanolamine, meglumine and L-arginine.

Another embodiment of the first aspect of the present invention is a pharmaceutical composition comprising one or more HMG-CoA reductase inhibitors, wherein the pH of the composition when dispersed in water is in the range of pH 7, 6, 5, 4 or lower. Preferably the pH of the composition when dispersed in water is in the range of pH 4-7, preferably pH 5-7, preferably pH 5.5-6.5. Surprisingly, a stable pharmaceutical composition has been prepared comprising one or more HMG-CoA reductase inhibitors, wherein the pH of the composition when dispersed in water is in the range of pH 7 or lower.

Another embodiment of the first aspect of the present invention is a pharmaceutical composition comprising one or more HMG-CoA reductase inhibitors, wherein the composition comprises less than 5% moisture, preferably less than 3%, preferably less than 2%, preferably less than 1%. Surprisingly, a stable pharmaceutical composition has been prepared comprising one or more HMG-CoA reductase inhibitors, wherein the composition comprises less than 5% moisture.

Another embodiment of the first aspect of the present invention is a pharmaceutical composition comprising:

(a) 5-25% of one or more HMG-CoA reductase inhibitors;

(b) 30-60% starch;

(c) 5-10% talc;

(d) 0.1-5% magnesium stearate; and

(e) 20-38% crospovidone.

Preferably the one or more HMG-CoA reductase inhibitors is present in an amount of 10-20%. Preferably the one or more HMG-CoA reductase inhibitors is fluvastatin, preferably fluvastatin sodium. Preferably the starch is present in an amount of 40-50%. Preferably the starch is maize starch, preferably low moisture maize starch. Preferably the talc is present in an amount of 6-8%. Preferably the magnesium stearate is present in an amount of 0.1-3%. Preferably the crospovidone is present in an amount of 25-35%. Surprisingly, a stable pharmaceutical composition has been prepared comprising one or more HMG-CoA reductase inhibitors, starch, talc, magnesium stearate and crospovidone.

Another embodiment of the first aspect of the present invention is a pharmaceutical composition comprising:

(a) 5-30% of one or more HMG-CoA reductase inhibitors;

(b) 70-90% lactose;

(c) 0.1-5% silica; and

(d) 0.1-5% magnesium stearate.

Preferably the one or more HMG-CoA reductase inhibitors is present in an amount of 10-20%. Preferably the one or more HMG-CoA reductase inhibitors is fluvastatin, preferably fluvastatin sodium. Preferably the lactose is present in an amount of 80-90%. Preferably the silica is present in an amount of 0.1-3%. Preferably the magnesium stearate is present in an amount of 0.1-3%. Surprisingly, a stable pharmaceutical composition has been prepared comprising one or more HMG-CoA reductase inhibitors, lactose, silica and magnesium stearate.

The pharmaceutical compositions of the current invention have enhanced stability over extended periods of time, e.g. whereby at least 95% of the initial amount of the active drug is still active after 2 years at ambient conditions. Preferably in the pharmaceutical compositions of the current invention, at least 99% of the initial amount of the active drug is still active after 2 years at ambient conditions. Even more preferably, in the pharmaceutical compositions of the current invention, at least 99.5% of the initial amount of the active drug is still active after 2 years at ambient conditions. Ambient conditions according to the ICH Guidelines are 25° C. and 60% relative humidity.

The meaning of ‘stable’ pharmaceutical composition as used herein means that after storage for six months at 40° C. and 75% relative humidity, no more than about 10%, preferably no more than about 5%, preferably no more than about 3%, preferably no more than about 2%, preferably no more than about 1%, and more preferably no more than about 0.5% of the HMG-CoA reductase inhibitor(s) has degraded.

In any of the embodiments of the first aspect of the invention, the pharmaceutical composition is preferably stable. Preferably the pharmaceutical composition does not include an alkaline agent. Preferably the pH of the composition when dispersed in water is in the range of pH 7, 6, 5, 4 or lower. Preferably the pH of the composition when dispersed in water is in the range of pH 4-7, preferably pH 5-7, preferably pH 5.5-6.5. Preferably the composition comprises less than 5% moisture, preferably less than 3%, preferably less than 2%, preferably less than 1%.

In preferred aspects of the current invention, the HMG-CoA reductase inhibitor(s) is selected from fluvastatin, pravastatin, lovastatin, simvastatin, atorvastatin, cerivastatin or rosuvastatin, or pharmaceutically acceptable salts thereof, or mixtures thereof. In a particularly preferred aspect of the current invention, the HMG-CoA reductase inhibitor is fluvastatin, preferably fluvastatin sodium.

The stable pharmaceutical composition of the invention can be a solution or suspension form, but is preferably a solid oral dosage form. Preferred dosage forms in accordance with the invention include tablets, capsules and the like which, optionally, may be coated if desired. Tablets can be prepared by conventional techniques, including direct compression, wet granulation and dry granulation. Capsules are generally formed from a gelatine material and can include a conventionally prepared granulate of excipients in accordance with the invention.

Preferably, the composition according to the first aspect of the invention is a solid oral dosage form, such as a tablet or a capsule. Most preferably, the composition according to the first aspect of the invention is a capsule.

The stable pharmaceutical composition of the invention typically comprises one or more conventional pharmaceutically acceptable excipient(s) selected from the group comprising a filler, a binder, a disintegrant, and a lubricant, and optionally further comprises at least one excipient selected from colouring agents, adsorbents, surfactants, film formers and plasticizers.

As described above, the stable pharmaceutical composition of the invention typically comprises one or more fillers such as microcrystalline cellulose, lactose, sugars, starches, modified starches, mannitol, sorbitol and other polyols, dextrin, dextran or maltodextrin; one or more binders such as lactose, starches, modified starch, maize starch, dextrin, dextran, maltodextrin, microcrystalline cellulose, sugars, polyethylene glycols, hydroxypropyl cellulose, hydroxypropyl methyl cellulose, ethyl cellulose, hydroxyethyl cellulose, methyl cellulose, carboxymethyl cellulose, gelatin, acacia gum, tragacanth, polyvinylpyrrolidone or crospovidone; one or more disintegrating agents such as croscarmellose sodium, cross-linked polyvinylpyrrolidone, crospovidone, cross-linked carboxymethyl starch, starches, microcrystalline cellulose, polyacrylin potassium; one or more different glidants or lubricants such as magnesium stearate, calcium stearate, zinc stearate, calcium behenate, sodium stearyl fumarate, talc, magnesium trisilicate, stearic acid, palmitic acid, carnauba wax or silicon dioxide.

If required, the stable pharmaceutical composition of the invention may also include surfactants and other conventional excipients. Typical surfactants that may be used are ionic surfactants such as sodium lauryl sulphate, or non-ionic surfactants such as different poloxamers (polyoxyethylene and polyoxypropylene copolymers), natural or synthesized lecithins, esters of sorbitan and fatty acids (such as Spano®), esters of polyoxyethylenesorbitan and fatty acids (such as Tween®), polyoxyethylated hydrogenated castor oil (such as Cremophor®), polyoxyethylene stearates (such as Brij®), dimethylpolysiloxane or any combination of the above mentioned surfactants.

Preferred excipients for the pharmaceutical compositions of the invention are starch such as maize starch, crospovidone, talc, magnesium stearate, lactose and silica. In particular the use of starch in combination with crospovidone has been found to be advantageous.

If the solid pharmaceutical formulation is in the form of coated tablets, the coating may be prepared from at least one film-former such as hydroxypropyl methyl cellulose, hydroxypropyl cellulose or methacrylate polymers, which optionally may contain at least one plasticizer such as polyethylene glycols, dibutyl sebacate, triethyl citrate, and other pharmaceutical auxiliary substances conventional for film coatings such as pigments, fillers and others.

A second aspect of the present invention provides a process for the preparation of a pharmaceutical composition according to the first aspect of the invention, comprising mixing one or more HMG-CoA reductase inhibitors with at least one pharmaceutically acceptable excipient. Preferably, the HMG-CoA reductase inhibitor(s) in the second aspect of the invention is selected from fluvastatin, pravastatin, lovastatin, simvastatin, atorvastatin, cerivastatin or rosuvastatin, or their pharmaceutically acceptable salts, or mixtures thereof. Most preferably, the HMG-CoA reductase inhibitor is fluvastatin, preferably fluvastatin sodium.

Preferably, the composition prepared in the second aspect of the invention is a solid oral dosage form, such as a tablet or a capsule. Most preferably, the composition prepared in the second aspect of the invention is a capsule.

A third aspect of the present invention provides the use of a pharmaceutical composition according to the first aspect of the invention for the preparation of a medicament for the treatment or prevention of hyperlipoproteinemia or atherosclerosis or related diseases.

The following paragraphs enumerated consecutively from 1 through 26 provide for various aspects of the present invention. In one embodiment, the present invention provides:

1. A stable pharmaceutical composition comprising one or more HMG-CoA reductase inhibitors, wherein the pharmaceutical composition does not include an alkaline agent.

2. A pharmaceutical composition comprising one or more HMG-CoA reductase inhibitors, wherein the pH of the composition when dispersed in water is in the range of pH 7 or lower.

3. A pharmaceutical composition comprising one or more HMG-CoA reductase inhibitors, wherein the composition comprises less than 5% moisture.

4. A pharmaceutical composition comprising:

(a) 5-25% of one or more HMG-CoA reductase inhibitors;

(b) 30-60% starch;

(c) 5-10% talc;

(d) 0.1-5% magnesium stearate; and

(e) 20-38% crospovidone.

5. A pharmaceutical composition comprising:

(a) 5-30% of one or more HMG-CoA reductase inhibitors;

(b) 70-90% lactose;

(c) 0.1-5% silica; and

(d) 0.1-5% magnesium stearate.

6. A pharmaceutical composition according to any one of the preceding paragraphs, wherein the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof.

7. A pharmaceutical composition according to paragraph 6, wherein the HMG-CoA reductase inhibitor is fluvastatin sodium.

8. A pharmaceutical composition according to any one of the preceding paragraphs, wherein the composition is a solid oral dosage form.

9. A pharmaceutical composition according to paragraph 8, wherein the composition is a tablet or a capsule.

10. A pharmaceutical composition according to paragraph 9, wherein the composition is a capsule.

11. A pharmaceutical composition according to any one of paragraphs 8 to 10, wherein the HMG-CoA reductase inhibitor is fluvastatin sodium.

12. A pharmaceutical composition according to any one of the preceding paragraphs, wherein the pharmaceutical composition is stable.

13. A pharmaceutical composition according to any one of the preceding paragraphs, wherein the pharmaceutical composition does not include an alkaline agent.

14. A pharmaceutical composition according to any one of the preceding paragraphs, wherein the pH of the composition when dispersed in water is in the range of pH 7 or lower.

15. A pharmaceutical composition according to any one of the preceding paragraphs, wherein the pH of the composition when dispersed in water is in the range of pH 4-7.

16. A pharmaceutical composition according to any one of the preceding paragraphs, wherein the composition comprises less than 5% moisture.

17. A process for the preparation of a pharmaceutical composition according to any one of the preceding paragraphs, comprising mixing one or more HMG-CoA reductase inhibitors with at least one pharmaceutically acceptable excipient.

18. A process according to paragraph 17, wherein the pharmaceutically acceptable excipient(s) does not include an alkaline agent.

19. A process according to paragraph 17 or 18, wherein the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof. 20. A process according to paragraph 18, wherein the HMG-CoA reductase inhibitor is fluvastatin sodium.

21. A process according to any one of paragraphs 17 to 20, wherein the composition is a solid oral dosage form.

22. A process according to paragraph 21, wherein the composition is a tablet or a capsule.

23. A process according to paragraph 22, wherein the composition is a capsule.

24. A process according to any one of paragraphs 21 to 23, wherein the HMG-CoA reductase inhibitor is fluvastatin sodium.

25. A pharmaceutical composition according to any one of paragraphs 1 to 16 for treating or preventing hyperlipoproteinemia or atherosclerosis or a related disease.

26. Use of a pharmaceutical composition according to any one of paragraphs 1 to 16 for the preparation of a medicament for the treatment or prevention of hyperlipoproteinemia or atherosclerosis or a related disease.

The present invention is illustrated, but in no way limited, by the following examples.

### EXAMPLES

### Comparative Example

Fluvastatin sodium was mixed with the following excipients in a conventional manner and filled into capsules.

| Component | Amount (mg) |
|---|---|
| Fluvastatin sodium | 42 |
| Pregelatinised maize starch | 84 |
| Talc | 19 |
| Magnesium stearate | 2 |
| Calcium carbonate | 126 |
| Sodium hydrogen carbonate | 4 |
| Microcrystalline cellulose | 114 |

The pH of the composition was >9.

### Example 1

Fluvastatin sodium was mixed with the following excipients in a conventional manner and filled into capsules.

| Component | Amount (mg) |
|---|---|
| Fluvastatin sodium | 44 |
| Low moisture maize starch | 129 |
| Talc | 19 |
| Magnesium stearate | 3 |
| Crospovidone | 85 |

The pH of the composition was 5.7-5.9. In a stability study at accelerated conditions (40° C. and 75% relative humidity), it was found after three months, the total level of impurities in the composition according to example 1 was 1.48% as compared to a total of 2.38% for the composition according to the comparative example.

### Example 2

Fluvastatin sodium was mixed with the following excipients in a conventional manner and filled into capsules.

| Component | Amount (mg) |
|---|---|
| Fluvastatin sodium | 44 |
| Lactose | 274 |
| Silica | 1.5 |
| Magnesium stearate | 2 |

The pH of the composition was 6.4. In a stability study at ambient conditions, it was found after five months, the total level of impurities in the composition according to example 2 was 0.11% as compared to a total of 0.2% for the composition according to the comparative example.

The stability data above illustrate that the compositions according to the present invention, at ambient and accelerated conditions, are more stable than the comparative example (a similar pharmaceutical composition stabilised by the inclusion of alkaline agents calcium carbonate and sodium hydrogen carbonate).

1-26. (canceled)

27. A stable pharmaceutical composition comprising one or more HMG-CoA reductase inhibitors, wherein the pharmaceutical composition does not include an alkaline agent.

28. A pharmaceutical composition comprising one or more HMG-CoA reductase inhibitors, wherein the pH of the composition when dispersed in water is in the range of pH 7 or lower.

29. A pharmaceutical composition comprising one or more HMG-CoA reductase inhibitors, wherein the composition comprises less than 5% moisture.

30. A pharmaceutical composition comprising:
(a) 5-25% of one or more HMG-CoA reductase inhibitors;
(b) 30-60% starch;
(c) 5-10% talc;
(d) 0.1-5% magnesium stearate; and
(e) 20-38% crospovidone.

31. A pharmaceutical composition comprising:
(a) 5-30% of one or more HMG-CoA reductase inhibitors;
(b) 70-90% lactose;
(c) 0.1-5% silica; and
(d) 0.1-5% magnesium stearate.

32. A pharmaceutical composition according to claim 27, wherein:
(a) the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof; and/or
(b) the HMG-CoA reductase inhibitor is fluvastatin sodium; and/or
(c) the composition is a solid oral dosage form; and/or
(d) the composition is a tablet or a capsule; and/or
(e) the composition is a capsule; and/or
(f) the pharmaceutical composition is stable; and/or
(g) the pharmaceutical composition does not include an alkaline agent; and/or
(h) the pH of the composition when dispersed in water is in the range of pH 7 or lower; and/or
(i) the pH of the composition when dispersed in water is in the range of pH 4-7; and/or
(j) the composition comprises less than 5% moisture.

33. A pharmaceutical composition according to claim 28, wherein:
(a) the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof; and/or
(b) the HMG-CoA reductase inhibitor is fluvastatin sodium; and/or
(c) the composition is a solid oral dosage form; and/or
(d) the composition is a tablet or a capsule; and/or
(e) the composition is a capsule; and/or
(f) the pharmaceutical composition is stable; and/or
(g) the pharmaceutical composition does not include an alkaline agent; and/or
(h) the pH of the composition when dispersed in water is in the range of pH 7 or lower; and/or
(i) the pH of the composition when dispersed in water is in the range of pH 4-7; and/or
(j) the composition comprises less than 5% moisture.

34. A pharmaceutical composition according to claim 29, wherein:
(a) the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof; and/or
(b) the HMG-CoA reductase inhibitor is fluvastatin sodium; and/or
(c) the composition is a solid oral dosage form; and/or
(d) the composition is a tablet or a capsule; and/or
(e) the composition is a capsule; and/or
(f) the pharmaceutical composition is stable; and/or
(g) the pharmaceutical composition does not include an alkaline agent; and/or
(h) the pH of the composition when dispersed in water is in the range of pH 7 or lower; and/or
(i) the pH of the composition when dispersed in water is in the range of pH 4-7; and/or
(j) the composition comprises less than 5% moisture.

35. A pharmaceutical composition according to claim 30, wherein:
(a) the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof; and/or
(b) the HMG-CoA reductase inhibitor is fluvastatin sodium; and/or
(c) the composition is a solid oral dosage form; and/or
(d) the composition is a tablet or a capsule; and/or
(e) the composition is a capsule; and/or
(f) the pharmaceutical composition is stable; and/or
(g) the pharmaceutical composition does not include an alkaline agent; and/or
(h) the pH of the composition when dispersed in water is in the range of pH 7 or lower; and/or
(i) the pH of the composition when dispersed in water is in the range of pH 4-7; and/or
(j) the composition comprises less than 5% moisture.

36. A pharmaceutical composition according to claim 31, wherein:
(a) the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof; and/or
(b) the HMG-CoA reductase inhibitor is fluvastatin sodium; and/or
(c) the composition is a solid oral dosage form; and/or
(d) the composition is a tablet or a capsule; and/or
(e) the composition is a capsule; and/or
(f) the pharmaceutical composition is stable; and/or
(g) the pharmaceutical composition does not include an alkaline agent; and/or
(h) the pH of the composition when dispersed in water is in the range of pH 7 or lower; and/or
(i) the pH of the composition when dispersed in water is in the range of pH 4-7; and/or
(j) the composition comprises less than 5% moisture.

37. A process for the preparation of a pharmaceutical composition according to claim 27, comprising mixing one or more HMG-CoA reductase inhibitors with at least one pharmaceutically acceptable excipient.

38. A process according to claim 37, wherein:
(a) the pharmaceutically acceptable excipient(s) does not include an alkaline agent; and/or
(b) the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof; and/or
(c) the HMG-CoA reductase inhibitor is fluvastatin sodium; and/or
(d) the composition is a solid oral dosage form; and/or
(e) the composition is a tablet or a capsule; and/or
(f) the composition is a capsule.

39. A process for the preparation of a pharmaceutical composition according to claim 28, comprising mixing one or more HMG-CoA reductase inhibitors with at least one pharmaceutically acceptable excipient.

40. A process according to claim 39, wherein:
(a) the pharmaceutically acceptable excipient(s) does not include an alkaline agent; and/or
(b) the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof; and/or
(c) the HMG-CoA reductase inhibitor is fluvastatin sodium; and/or
(d) the composition is a solid oral dosage form; and/or
(e) the composition is a tablet or a capsule; and/or
(f) the composition is a capsule.

41. A process for the preparation of a pharmaceutical composition according to claim 29, comprising mixing one or more HMG-CoA reductase inhibitors with at least one pharmaceutically acceptable excipient.

42. A process according to claim 41, wherein:
(a) the pharmaceutically acceptable excipient(s) does not include an alkaline agent; and/or
(b) the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof; and/or
(c) the HMG-CoA reductase inhibitor is fluvastatin sodium; and/or
(d) the composition is a solid oral dosage form; and/or
(e) the composition is a tablet or a capsule; and/or
(f) the composition is a capsule.

43. A process for the preparation of a pharmaceutical composition according to claim 30, comprising mixing one or more HMG-CoA reductase inhibitors with at least one pharmaceutically acceptable excipient.

44. A process according to claim 43, wherein:
(a) the pharmaceutically acceptable excipient(s) does not include an alkaline agent; and/or
(b) the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof; and/or
(c) the HMG-CoA reductase inhibitor is fluvastatin sodium; and/or
(d) the composition is a solid oral dosage form; and/or
(e) the composition is a tablet or a capsule; and/or
(f) the composition is a capsule.

45. A process for the preparation of a pharmaceutical composition according to claim 31, comprising mixing one or more HMG-CoA reductase inhibitors with at least one pharmaceutically acceptable excipient.

46. A process according to claim 45, wherein:
(a) the pharmaceutically acceptable excipient(s) does not include an alkaline agent; and/or
(b) the HMG-CoA reductase inhibitor(s) is selected from fluvastatin, pravastatin, lovastatin, simvastatin, atorvastatin, cerivastatin or rosuvastatin, or a pharmaceutically acceptable salt thereof, or a mixture thereof; and/or
(c) the HMG-CoA reductase inhibitor is fluvastatin sodium; and/or
(d) the composition is a solid oral dosage form; and/or
(e) the composition is a tablet or a capsule; and/or
(f) the composition is a capsule.

47. A method of treating or preventing hyperlipoproteinemia or atherosclerosis or a related disease, the method comprising administering to a patient in need thereof a therapeutically or prophylactically effective amount of a pharmaceutical composition according to claim 27.

48. A method of treating or preventing hyperlipoproteinemia or atherosclerosis or a related disease, the method comprising administering to a patient in need thereof a therapeutically or prophylactically effective amount of a pharmaceutical composition according to claim 28.

49. A method of treating or preventing hyperlipoproteinemia or atherosclerosis or a related disease, the method comprising administering to a patient in need thereof a therapeutically or prophylactically effective amount of a pharmaceutical composition according to claim 29.

50. A method of treating or preventing hyperlipoproteinemia or atherosclerosis or a related disease, the method comprising administering to a patient in need thereof a therapeutically or prophylactically effective amount of a pharmaceutical composition according to claim 30.

51. A method of treating or preventing hyperlipoproteinemia or atherosclerosis or a related disease, the method comprising administering to a patient in need thereof a therapeutically or prophylactically effective amount of a pharmaceutical composition according to claim 31.
